(19) United States
(12) Patent Application Publication
(10) Pub. No.: US 2020/0317561 A1
(43) Pub. Date: Oct. 8, 2020

(54) Process For The Preparation Of Multi-Coloured Glass Ceramic Blanks

(71) Applicant: Ivoclar Vivadent AG, Schaan (LI)

(72) Inventors: Lars Arnold, Gams (CH); Harald Bürke, Frastanz (AT); Alexander Engels, Feldkirch (AT)

(21) Appl. No.: 16/833,961

(22) Filed: Mar. 30, 2020

(30) Foreign Application Priority Data
Apr. 4, 2019 (EP) 19167345.8

(51) Int. Cl. C03C 10/00; C03C 4/02; C03C 4/12; A61K 6/802; C03B 32/02; C03B 19/06

(52) U.S. Cl. CPC C03C 10/0027; C03C 4/02; C03C 4/12; C03C 2204/00; C03B 32/02; C03B 19/063; A61K 6/802

(57) ABSTRACT

A process for the preparation of multi-coloured glass ceramic blanks for dental purposes is described, in which lithium silicate glasses with different compositions are introduced into a mould in order to form a glass blank, the glass blank is optionally compacted by pressing, the glass blank is heat-treated in order to obtain a glass ceramic blank with lithium silicate as main crystal phase, and the glass ceramic blank is compacted by hot pressing.

### CROSS-REFERENCE TO RELATED APPLICATIONS

This application claims priority to European patent application No. 19167345.8 filed on Apr. 4, 2019, the disclosure of which is incorporated herein by reference in its entirety.

### TECHNICAL FIELD

The invention relates to a process by which multi-coloured glass ceramic blanks can be produced in a simple manner which can imitate the optical properties of natural tooth material very well and which are suitable in particular for the simple production of aesthetically demanding dental restorations with very good optical and mechanical properties.

### BACKGROUND

Creating blanks which satisfy the various requirements for use in the field of dental technology represents a major challenge. Such blanks should not only be simple to produce, but should also be simple to shape to the desired dental restorations and still yield high-strength restorations. Finally, the blanks should already have such a structure that the restorations produced from them have optical properties which come very close to those of natural tooth material, so that a subsequent expensive veneering of the restorations can be dispensed with. This is because natural teeth are not single-coloured, but they have a complex colouring, in that different regions of the same tooth generally differ from each other in terms of their colour and their translucence.

Blanks for use in dental technology are known from the state of the art.

DE 103 36 913 A1 and corresponding U.S. Pat. Nos. 7,316,740 and 8,042,358, which U.S. patents are hereby incorporated by reference in their entirety, describe blanks based on lithium metasilicate glass ceramic, which are produced by heat treatment of glass blanks from cast starting glass, so-called solid glass blanks or monolithic glass blanks. This procedure is therefore also referred to as “solid glass technology”. The blanks produced can be machined in a simple manner because of their relatively low strength and, through further heat treatment, can be converted into high-strength dental restorations based on lithium disilicate glass ceramic. The blanks produced are, however, only single-coloured blanks, which thus also only result in single-coloured dental restorations. To effect multi-colouration, an expensive subsequent veneering of the dental restorations prepared is therefore also required.

H. Zhang et al. describe, in J. Am. Ceram. Soc. 98; 3659-3662 (2015), the production of a lithium metasilicate glass ceramic by hot pressing of a special glass powder at 760° C. for 30 min using a pressure of 30 MPa. In the process, obviously predominantly surface crystallization takes place, which is probably a reason for the low flexural strength of the lithium disilicate glass ceramic obtained from this glass ceramic through further heat treatment at 855°.

WO 2014/124879 and corresponding U.S. Pat. No. 10,064,708, which is hereby incorporated by reference, describe multi-coloured lithium silicate blanks, which have differently coloured monolithic layers. For the production thereof, layers of differently coloured solid glass are joined to each other, e.g. by pouring onto an existing solid-glass layer the melt of a glass of another colour, followed by a heat treatment. For a good match to the optical properties of natural tooth material to be replaced, it is however necessary to provide a whole series of differently coloured solid-glass layers, which is very expensive and time-consuming when using the described procedures. Moreover, it is impossible to imitate a continuous colour gradient in this way.

### SUMMARY OF THE INVENTION

According to the invention, the described problems with the conventional processes are to be avoided. The object of the invention is in particular to provide a process whereby multi-coloured glass ceramic blanks can be produced in a simple manner by which the optical properties of natural tooth material can be imitated very well, which can be given the shape of the ultimately desired dental restoration by machining in a simple manner and which, after shaping, can be transformed into dental restorations with excellent mechanical and optical properties.

This object is achieved by the process according to the claims. A subject of the invention is likewise a multi-coloured glass ceramic blank, use of the glass ceramic blank as well as a process for the preparation of a dental restoration.

### DETAILED DESCRIPTION

The process according to the invention for the preparation of a multi-coloured glass ceramic blank for dental purposes with lithium silicate as main crystal phase is characterized in that
(a) (i) differently coloured powders of lithium silicate glasses or (ii) suspensions of differently coloured powders of lithium silicate glasses in liquid media are introduced into a mould to form a glass blank,
(b) optionally the glass blank from step (a) is compacted by pressing,
(c) the glass blank from step (a) or (b) is heat treated to obtain a glass ceramic blank with lithium silicate as main crystal phase, and
(d) the glass ceramic blank from step (c) is compacted by hot pressing.

Surprisingly, the process according to the invention allows the production of a glass ceramic blank which can be made multi-coloured in a very simple manner, and which allows the production of dental restorations which not only simulate the optical properties of natural tooth material and can in particular have continuous colour gradients, but at the same time also have excellent mechanical properties.

The multi-coloured nature of the glass ceramic blank prepared according to the invention means that it has regions with different compositions which, during transformation of the blank into the desired dental restoration by means of heat treatment, result in regions with different colours and thus make the dental restoration multi-coloured. By differences in the colour are also meant differences in the translucence, opalescence and/or fluorescence.

The colour can be determined in particular via the Lab value or with the aid of a shade guide customary in the dental industry.

The translucence can be determined in particular via the contrast ratio (CR value) according to British Standard BS 5612.

The opalescence can be determined by means of photometric measurement, in particular as described in WO 2014/209626 and corresponding U.S. Pat. No. 10,004,668, which is hereby incorporated by reference in its entirety.

The fluorescence can be determined in particular by means of fluorescence spectrometers, e.g. an FL1039-type fluorescence spectrometer, using a PMT 1424M-type photomultiplier detector, both from Horiba Jobin Yvon GmbH.

In step (a) of the process according to the invention, in a first variant (i), differently coloured powders of lithium silicate glasses or, in a second variant (ii), suspensions of differently coloured powders of lithium silicate glasses in liquid media are introduced into a mould in order to form a glass blank.

By differently coloured powders, in both variants (i) and (ii), are meant powders with different compositions which, during further processing via the glass ceramic blank prepared according to the invention to form the dental restoration, produce regions with different colours in the dental restoration. This desired multi-coloured nature of the dental restoration can be in particular a continuously changing colour, such as a colour gradient and/or translucence gradient. Such gradients of colour and translucence normally occur in natural tooth material, e.g. between dentine and incisal edge.

The different colouring of the lithium silicate glass powders used in both variants (i) and (ii) can be produced through the different composition of the lithium silicate glasses or also by admixing additives, such as colour components and/or fluorescence components, into the glasses. This represents a particular advantage of the process according to the invention, as in this way a different colouring of the powders can be achieved not only through components of the glasses, such as colouring ions, but also by adding pigments, such as colour pigments and/or fluorescence pigments, to the glasses. By contrast, in the case of the use of so-called solid glass technology, i.e. the use of cast monolithic glass blanks, coloration is only possible by ion colouring.

For the production of the lithium silicate glasses, a mixture of suitable starting materials, such as carbonates, oxides, phosphates and fluorides, is usually first melted at temperatures of in particular from 1300 to 1600° C. for 1 to 10 h. The glass melt obtained is then poured into water in order to produce a glass frit. In order to achieve a particularly high degree of homogeneity, the glass frit can be melted again and the glass melt obtained can be transformed into a glass frit again by being poured into water. Finally, the glass frit is crushed to powder with the desired particle size. Mills suitable for this are, for example, roller mills, ball mills or opposed jet mills. Additives can then also be added to the powders obtained in order to produce the different powders for the first variant (i) and the second variant (ii).

The powders of the first variant (i) can, for example, contain colour and/or fluorescence pigments, such as ceramic pigments, pressing agents and in particular binders, as additives. The binders serve for the cohesion of the powder particles and they therefore promote the obtaining of a stable glass blank. Preferred examples of binders are polyvinyl alcohols and cellulose derivatives, such as sodium carboxymethyl cellulose. Polyethylene glycols or stearates are preferably used as pressing agents.

In addition to the lithium silicate glass, the powders of the first variant (i) usually contain up to 10 wt.-% additives. Colour and/or fluorescence pigments are typically used in an amount of from 0 to 5 wt.-%, pressing agents are typically used in an amount of from 0 to 3 wt.-%, preferably 0 to 1 wt.-%, and binders are typically used in an amount of from 0 to 5 wt.-%, preferably 0.3 to 3 wt.-%, as preferred additives.

The powders of the suspensions used in the second variant (ii) can contain colour and/or fluorescence pigments as additives, as previously indicated by their type and amount for the variant (i).

For the production of the suspensions, these powders are usually suspended in liquid media and in particular aqueous media. The liquid media preferably contain auxiliary agents, such as binders, dispersants, in particular in an amount of from 0 to 3 wt.-%, viscosity-adjusting agents, in particular in an amount of from 0 to 3 wt.-%, and pH-adjusting agents, in particular in an amount of from 0 to 1 wt.-%, preferably 0.001 to 0.5 wt.-%.

Preferred binders are polyvinyl alcohols and cellulose derivatives, such as sodium carboxymethyl cellulose. Polymers and lecithins are examples of suitable dispersants. Xanthan gums and starches are examples of suitable viscosity-adjusting agents. Inorganic or organic acids, such as acetic acid and hydrochloric acid, are examples of suitable pH-adjusting agents.

The suspensions contain in particular 30 to 90 wt.-% and preferably 40 to 70 wt.-% powder.

In step (a) of the process according to the invention, at least two differently coloured powders (i) or at least two suspensions of differently coloured powders (ii) are used.

The different powders (i) or the different suspensions (ii) are introduced into the mould in a suitable manner in order to effect the desired multi-coloured nature in the glass ceramic blank and the dental restoration produced therefrom and in particular to effect a desired progression of colour, translucence and/or fluorescence. This is usually achieved in that the different powders (i) or the different suspensions (ii) are introduced into the mould in a controlled manner. For example, through suitably controlled mixing of either different powders or different suspensions, a continuous change in the composition of the mixture introduced into the mould can be achieved, and thus a continuous colour gradient can be generated. For example, two or more different powders can be introduced into the mould such that initially only the first powder is added, to which a steadily increasing proportion of at least one further powder is gradually added.

In a preferred embodiment of the process according to the invention, the powders (i) or suspensions (ii) are introduced into the mould in such a way that the multi-coloured glass ceramic blank produced has a continuously changing colour. With such a glass ceramic blank, the colour gradient of natural tooth material can be simulated particularly well.

In a preferred embodiment of the process according to the invention, in step (a) different powders (i) are introduced into the mould and the optional step (b) is carried out. The pressing according to step (b) leads to the glass blank having a good strength. The pressed glass blank is also referred to as a powder compact, as it consists of pressed powder particles.

In another preferred embodiment of the process according to the invention, in step (a) different suspensions (ii) are introduced into the mould and the liquid media are removed.

The glass blank obtained after removal of the liquid media is as a rule additionally subjected to a drying process at 10 to 100° C. It is a particular advantage of this embodiment that the glass blank obtained thereafter also normally has sufficient strength for further processing even without further compaction through pressing.

The suspensions (ii) are usually introduced into a mould which has pores. The introduction is usually effected by pouring in. The pores are openings through which the liquid medium can at least partially be removed from the suspensions, with the result that the powder particles can be deposited in the mould and can finally form the glass blank.

Typically, substantially all of the liquid medium is removed via the pores. However, it is also possible that remaining liquid not removed via the pores is poured or suctioned out of the mould. This is usually the case when a sufficiently thick layer of powder particles has already been deposited.

The mould can for example be one of the moulds usually employed for slip casting or pressure casting processes. These are in particular moulds with a wall made of gypsum through which, because of the capillary action of the gypsum pores, liquid medium such as water can be removed from the suspension. However, moulds made of plastic, ceramic or metal can also be used, which already have pores or in which pores are provided e.g. by providing them with filter elements, such as membrane filters, paper filters and sintered filters.

The mould used is in particular comprised of several parts in order to facilitate the simple removal of the blank formed from the mould. In a particularly preferred embodiment the mould has connections via which pressure, for example by means of compressed air, can be exerted on the introduced suspension and/or a negative pressure can be applied to the pores. Both measures serve to speed up the removal of the liquid medium from the mould and thus to shorten the process. With the help thereof, a very quick and thus economical production of glass blanks is possible, which is particularly advantageous in particular in the case of manufacturing on an industrial scale.

Moreover, the removal of the liquid medium can also be effected by lyophilization. For this, the glass blank produced by slip casting is cooled in a dense but flexible mould, for example made of silicone, to temperatures at which the liquid components of the suspension freeze. Through subsequent sublimation of these liquid components at reduced pressure, the removal thereof and thus the complete drying is effected. A separate heat treatment for drying the blank is then normally no longer necessary.

In a further preferred embodiment of the process according to the invention, the powders (i) have a particle size of from 0.5 to 150 μm, in particular 1 to 100 μm, and the powders of the suspensions (ii) have a particle size of from 0.5 to 80 μm, in particular 0.5 to 70 μm, measured by laser diffraction according to ISO 13320 (2009). The samples used to determine the particle size were produced in particular according to DIN ISO 14887 (2010), wherein water was used as solvent in order to disperse the samples.

The average particle size as d₅₀ value of the powders (i) and (ii) is 5 to 30 μm, preferably 10 to 20 μm, determined on the basis of the proportions by volume measured by laser diffraction according to ISO 13320 (2009).

The glass blank formed in step (a) is usually in the shape of a block, cylinder or disc, as blanks of such geometry can easily be given the shape of the desired dental restoration in usual processing machines. The blank can also already have a holding device formed in one piece with the blank, such as a holding pin, which makes the later attachment thereof, for example by gluing, unnecessary.

Therefore, the mould employed also usually has a geometry which allows the production of such blanks. The mould can be in one piece or, for easier removal of the produced blank, also be comprised of several pieces, in particular 3 pieces.

In the optional step (b) of the process according to the invention the glass blank from step (a) is compacted by pressing. It is preferred that the powders of the first variant (i) used in step (a) are subjected to this optional step.

It is further preferred that the pressing in step (b) is effected at a temperature of less than 60° C., preferably at 15 to 35° C., and in particular at a pressure of from 20 to 120 MPa, preferably 50 to 120 MPa. The pressing normally takes place at room temperature, and is therefore also referred to as cold pressing.

In step (c) of the process according to the invention the glass blank from step (a) and (b) is heat treated in order to obtain a glass ceramic blank with lithium silicate as main crystal phase. It is preferred to carry out the heat treatment at a temperature of less than 700° C., preferably 550 to 690° C., and for a period of in particular from 2 to 60 min, preferably 5 to 30 min.

The glass blank is, before the heat treatment, usually subjected to debinding at temperatures of in particular from 400 to 450° C. in order to remove any binders or other organic additives that may be present. The glass blank is then normally heated directly to the temperature of the heat treatment. Heating to the temperature of the heat treatment and maintaining this temperature effects the formation of nuclei and the crystallization of lithium silicate, in particular of lithium metasilicate, as main crystal phase.

In step (d) of the process according to the invention the glass ceramic blank from step (c) is compacted by hot pressing. This compaction surprisingly succeeds in giving the glass ceramic blank a density which does not differ substantially from the density of a glass ceramic blank produced in a conventional manner by the solid glass technology. While glass powders and thus the so-called “powder technology” are used in the process according to the invention to produce the glass ceramic blank, in solid glass technology glass melts are poured into a mould in order to form a monolithic glass blank which after heat treatment yields the desired glass ceramic blank.

The hot pressing in step (d) is preferably carried out in a mould to which glass does not adhere. Suitable materials for such a mould are materials based on carbon and ceramics based on nitrides. Metallic materials are likewise suitable, if a suitable release agent is placed between the mould and the glass ceramic blank to be compacted.

It is preferred that the hot pressing is effected at a temperature of from 650 to 780° C., in particular 700 to 750° C., and at a pressure of in particular from 5 to 50 MPa, preferably 10 to 30 MPa.

It is further preferred that the hot pressing is effected for a period of from 0.1 to 10 min, preferably 0.3 to 5 min. A further advantage of the process according to the invention is that a hot pressing for such a short period is sufficient to produce a glass ceramic blank from which dental restorations with excellent optical and mechanical properties can be produced in a quick and easy manner.

In a further preferred embodiment the hot pressing is effected at an atmospheric pressure of less than 0.1 bar and preferably at 0.01 to 0.08 bar.

The multi-coloured glass ceramic blank obtained after the hot pressing preferably has a density of from 2.4 to 2.6 and in particular 2.44 to 2.56 g/cm³. The determination of the density of this blank was effected in deionized water according to the Archimedes' principle. The glass ceramic blank according to the invention thus surprisingly has a similar density to a corresponding conventional blank produced by solid glass technology.

In the case of the glass blanks and glass ceramic blanks obtained after steps (a), (b) and (c) the determination of the mass was effected by weighing and the determination of the volume was effected by optical measurement by means of the strip projection process (ATOS 3D scanner from GOM GmbH, Germany). The density was then calculated according to the formula p=mass/volume.

The multi-coloured glass ceramic blank obtained has in particular lithium metasilicate as main crystal phase. It is further preferred that the glass ceramic blank has less than 20 wt.-%, in particular less than 10 wt.-%, preferably less than 5 wt.-% and even more preferred less than 3 wt.-% lithium disilicate, as larger amounts of lithium disilicate crystals can impair the shaping by means of machining.

The term “main crystal phase” denotes the crystal phase which has the highest proportion by mass of all the crystal phases present in the glass ceramic. The masses of the crystal phases are in particular determined using the Rietveld method. A suitable process for the quantitative analysis of the crystal phases by means of the Rietveld method is described e.g. in M. Dittmer's doctoral thesis “Glaser and Glaskeramiken im System MgO—Al₂O₃—SiO₂ mit ZrO₂ als Keimbildner” [Glasses and glass ceramics in the MgO—Al₂O₃—SiO₂ system with ZrO₂ as nucleating agent], University of Jena 2011.

In a preferred embodiment, the glass ceramic blank contains more than 10 wt.-%, preferably more than 20 wt.-% and particularly preferably more than 25 wt.-% lithium metasilicate crystals.

The multi-coloured glass ceramic blank contains in particular at least one and preferably all of the following components in the amounts indicated:

| Component | wt.-% |
|---|---|
| SiO₂ | 64.0 to 75.0, preferably 64.0 to 72.0 |
| Li₂O | 13.0 to 17.0, preferably 13.5 to 16.0 |
| K₂O | 0 to 5.0, preferably 3.0 to 5.0 |
| Al₂O₃ | 0.5 to 5.0, preferably 1.5 to 3.5 |
| P₂O₅ | 2.0 to 5.0, preferably 2.5 to 4.0 |

In a further preferred embodiment, the multi-coloured glass ceramic blank contains at least one and preferably all of the following components in the amounts indicated:

| Component | wt.-% |
|---|---|
| SiO₂ | 64.0 to 75.0 |
| Li₂O | 13.0 to 17.0 |
| K₂O | 0 to 5.0 |
| Al₂O₃ | 0.5 to 5.0 |
| P₂O₅ | 2.0 to 5.0 |
| ZrO₂ | 0 to 5.0 |
| MgO | 0 to 5.0 |
| SrO | 0 to 5.0 |
| ZnO | 0 to 5.0 |
| F | 0 to 1.0 |
| Colouring and/or | 0 to 10.0, preferably 0 to 7.0 |
| fluorescent components | |

wherein the colouring and/or fluorescent components are in particular selected from the group of oxides of Sn, Ce, V, Mn, Co, Ni, Cu, Fe, Cr, Tb, Eu, Er and Pr.

The invention is likewise directed to a multi-coloured glass ceramic blank which is obtainable according to the process of the invention. Compared with blanks which have been obtained by solid glass technology, not only is the blank according to the invention characterized by the much simpler possibility for generating multi-colouration, but it can also be machined in a shorter time and with less tool wear. This is a particularly important advantage in the very quick and cost-effective production of highly aesthetic dental restorations desired today.

Because of the described particular properties of the blank according to the invention, it is suitable in particular for use in dentistry and in particular as a dental material and preferably for the preparation of dental restorations. The invention therefore also relates to the use of the blank as a dental material and preferably for the production of dental restorations and in particular of crowns, abutments, abutment crowns, inlays, onlays, veneers, facets, bridges and caps.

The invention is finally also directed to a process for the preparation of a dental restoration, in which
(i) the described process according to the invention is carried out to produce a multi-coloured glass ceramic blank,
(ii) the multi-coloured glass ceramic blank is given the shape of the dental restoration by machining, and
(iii) at least one heat treatment at a temperature of more than 750° C., preferably 800 to 900° C., is carried out.

The machining in step (ii) is usually effected by material-removal processes and in particular by milling and/or grinding. It is preferred that the machining is effected with computer-controlled milling and/or grinding devices. Such devices are known to a person skilled in the art and are also customary in the trade.

In a preferred embodiment of the process, the heat treatment in step (iii) effects the formation of lithium disilicate as main crystal phase. Glass ceramics with lithium disilicate as main crystal phase are characterized by excellent mechanical properties, such as are required for a material which is to replace natural tooth material. In the glass ceramic produced by the heat treatment the crystals and in particular the lithium disilicate crystals are surprisingly very homogeneously distributed, although the glass ceramic was not produced using the so-called solid glass technology, i.e. using cast monolithic glass blocks.

After step (iii) has been carried out, there is a dental restoration which has very good mechanical properties and a high chemical stability. In addition, because of its multi-coloured nature, it allows an excellent imitation of the optical properties of natural tooth material, e.g. of colour gradients from the dentine to the cutting edge.

It is preferred that the dental restoration obtained has a biaxial flexural strength σ_{B} of at least 300 MPa, in particular 360 to 600 MPa, determined according to ISO 6872:2008 (piston-on-three-ball test) and/or a fracture toughness K_{1c} of at least 2.0 MPa m^{1/2}, in particular 2.1 to 2.5 MPa m^{1/2}, determined according to ISO 6872:2008 (SEVNB method).

Finally, the dental restoration can also be produced from the glass ceramic blanks according to the invention without substantial shrinkage. This is based in particular on the fact that the blanks according to the invention have a high density of in particular from 2.4 to 2.6, preferably 2.44 to 2.56, and accordingly have only a very low porosity. In this they differ from the glass ceramic blanks produced in the usual way by means of powder technology, which normally have a high porosity. Through the use of the blanks according to the invention, therefore, dental restorations with precisely the desired dimensions can be produced in a particularly simple manner.

The dental restoration produced by means of the process according to the invention is preferably selected from the group of crowns, abutments, inlays, onlays, veneers, facets, bridges and caps.

The invention is described in further detail in the following with reference to examples.

### EXAMPLES

The examples explain in particular the production of multi-coloured glass ceramic blocks with a gradient of colour and translucence, and the use thereof for the production of dental restorations.

### Examples 1 to 9

### A. Preparation of Lithium Silicate Glasses

First, nine different lithium silicate glasses with the composition indicated in Table I were produced, wherein the glasses were used to simulate either the dentine or the tooth cutting edge. The additions likewise indicated in Table I were added to these glasses.

To produce these glasses, a mixture of corresponding raw materials was first melted at 1500° C. for a period of 1.5 h, wherein the melting was very easily possible without the formation of bubbles or streaks. In each case a glass frit was produced by pouring the melt obtained into water.

### B. Preparation of Single-Coloured Glass Ceramic Blocks for Determining Properties

These glass frits were first crushed in an FM 2/2 roller mill (Merz Aufbereitungstechnik GmbH, Germany) to a size of <3 mm and then crushed further in an AFG 100 opposed jet mill (Hosokawa Alpine AG) to a size of 15 μm (d₅₀ value) to produce glass powders in each case.

The glass powders obtained were granulated using a GPCG 3.1 spray granulator (Glatt GmbH Germany) by spraying aqueous suspensions with 1.0 wt.-% binder and 0.5 wt.-% pressing agents onto the glass powders in a fluidized bed.

The granulated glass powders were then introduced into a 3-part steel mould consisting of die plate as well as top punch and bottom punch to produce a single-coloured glass blank in each case.

These single-coloured glass blanks were compacted in an isostatic press at room temperature by pressing at the pressure indicated in Table II.

The compacted blanks were debinded in an N11/HR sintering furnace (Nabertherm GmbH, Germany) under the conditions indicated in Table II and crystallized to form glass ceramic blanks with lithium metasilicate as main crystal phase. These glass ceramic blanks were then hot-pressed in a DSP 515 pressure-sintering press (Dr. Fritsch Sondermaschinenbau GmbH, Germany) under the conditions likewise indicated in Table II. The density of the glass ceramic blanks obtained was measured according to the Archimedes' principle and their lithium metasilicate content was also determined by X-ray diffraction examinations using Rietveld analysis. The values obtained are listed in Table II.

Finally, these blanks were crystallized further under the conditions also indicated in Table II to produce lithium disilicate as main crystal phase. The lithium disilicate blanks produced in this way had the properties likewise indicated in Table II. The biaxial flexural strength σ_{B} was measured according to ISO 6872:2015 (piston-on-three-ball test) and the fracture toughness K_{1c} was determined according to ISO 6872:2015 (SEVNB method). To determine the Lab values and the contrast ratio (CR value), a CM-3700d spectrophotometer from Konica Minolta was used, wherein the contrast ratio was determined according to British Standard BS 5612. The density was determined according to the Archimedes' principle and the lithium metasilicate content and lithium disilicate content were measured by X-ray diffraction examinations using Rietveld analysis.

The flexural strength of these lithium disilicate blanks produced by powder technology was surprisingly very high and comparable to that of lithium disilicate glass ceramic which was produced in the conventional way by solid glass technology and normally has a strength of at least 360 MPa.

The fracture toughness of the lithium disilicate blanks was also surprisingly perfectly comparable to that of lithium disilicate glass ceramic which was produced in the conventional way by solid glass technology, and typically has a fracture toughness of from 2.2 to 2.3 MPa m^{1/2}.

### C. Preparation of Multi-Coloured Glass Ceramic Blocks

### a) Gradient Blocks of Glass Powders According to Examples 1 and 2

To produce multi-coloured glass ceramic blocks, granulated glass powder according to Example 1 was used to simulate dentine and granulated glass powder according to Example 2 was used to simulate the cutting edge.

These granulated powders were produced in the same way as explained under B. above. The powders were then introduced into a three-part steel mould mentioned under B. using a device for gradual dosing and mixing in such a way that glass blanks with gradual colour and translucence progression were produced. Then, the glass blanks were transformed into glass ceramic blocks with lithium metasilicate as main crystal phase in the way explained above under B.

The multi-coloured glass ceramic blocks obtained were machined to form crowns in a CAD/CAM unit. For this, the blocks were provided with a suitable holder, and then given the desired shape in an inLab MC XL grinding unit (Sirona Dental GmbH, Germany). For the processing of the blocks, the same grinding parameters could be used as for commercial e.max CAD blocks, Ivoclar Vivadent, Liechtenstein.

The machinability of the glass ceramic blocks was tested in comparison with commercial glass ceramic blocks of the e.max CAD LT type, Ivoclar Vivadent AG, Liechtenstein, which were produced by solid glass technology. The tool life was likewise tested. For this, always the same molar crown was ground out of blocks with the same dimensions provided with holders on the inLab MC XL grinding unit and the time from the start to the process end was determined. For the tool life, the number of crowns which could be produced until the unit indicated the need for the first tool change was determined.

It was shown that the glass ceramic blocks according to the invention are superior to the commercial blocks, in that they could be machined at least 10% more quickly and the tool life was at least 35% longer.

These favourable properties predestine the glass ceramic blocks according to the invention for very quickly supplying patients with a dental restoration which meets very high demands in terms of both their optical properties and their mechanical properties.

### b) Gradient Blocks of Glass Powders According to Examples 3 and 4

Multi-coloured glass ceramic blocks were produced in the same way as described above under a), wherein the only difference is that glass powder according to Example 3 was used to simulate the dentine and glass powder according to Example 4 was used to simulate the cutting edge.

These glass ceramic blocks were also clearly superior to commercial blocks, in that they could be machined more quickly and the tool life was longer.

These favourable properties were also displayed by glass ceramic blocks which had been produced analogously to a) and b) in which, however, at least one of the glass powders used had been replaced by another glass powder listed in Table I.

### D. Heat Treatment for the Production of Dental Restorations

The machined blocks obtained under C. were then subjected to a heat treatment at 840° C. for a period of 7 min. Then, the crowns obtained were slowly cooled to room temperature and an X-ray diffraction examination revealed that they had lithium disilicate as main crystal phase.

The crowns obtained had a high strength. Moreover, they showed a continuous gradient of colour and translucence from dentine to cutting edge, and thus they simulated the optical properties of natural tooth material in an excellent way.

The average value for the flexural strength of 12 examined samples was 403.82 MPa with a standard deviation of 55.16 MPa for lithium disilicate blocks which had been produced from the gradient block according to a) (powders according to Examples 1 and 2). The average value for the fracture toughness of 6 examined samples of these lithium disilicate blocks was 2.27 MPa m^{1/2} with a standard deviation of 0.15 MPa m^{1/2}.

The average value for the flexural strength of 12 examined samples was 470.57 MPa with a standard deviation of 100.66 MPa for lithium disilicate blocks which had been produced from the gradient blocks according to b) (powders according to Examples 3 and 4).

TABLE I

| | Example | | | | | | | | |
|---|---|---|---|---|---|---|---|---|---|
| | 1 | 2 | 3 | 4 | 5 | 6 | 7 | 8 | 9 |
| | Use as | | | | | | | | |
| | | cutting | | cutting | | | | cutting | |
| | dentine | edge | dentine | edge | dentine | dentine | dentine | edge | dentine |
| | wt.-% | wt.-% | wt.-% | wt.-% | wt.-% | wt.-% | wt.-% | wt.-% | wt.-% |
| Composition of | | | | | | | | | |
| the glass | | | | | | | | | |
| SiO₂ | 66.736 | 67.770 | 67.098 | 67.433 | 68.479 | 67.100 | 66.307 | 68.288 | 66.693 |
| K₂O | 4.222 | 4.190 | 4.211 | 4.169 | 4.297 | 4.211 | 4.147 | 4.222 | 4.123 |
| SrO | 2.861 | 2.870 | 2.864 | 2.856 | 2.923 | 2.864 | 2.825 | 2.892 | 2.824 |
| Li₂O | 14.706 | 14.530 | 14.644 | 14.458 | 14.946 | 14.645 | 14.412 | 14.641 | 14.299 |
| Al₂O₃ | 3.001 | 2.000 | 2.651 | 1.990 | 2.705 | 2.651 | 2.465 | 2.015 | 1.968 |
| P₂O₅ | 3.601 | 3.600 | 3.601 | 3.582 | 3.675 | 3.601 | 3.550 | 3.628 | 3.543 |
| MgO | — | 0.400 | 0.140 | 0.398 | 0.100 | 0.140 | 0.197 | 0.405 | 0.394 |
| ZrO₂ | — | 1.670 | 0.585 | 2.159 | 0.501 | 0.350 | 0.823 | 1.823 | 2.136 |
| ZnO | 1.970 | 1.950 | 1.963 | 1.940 | 2.003 | 1.963 | 1.932 | 1.965 | 1.919 |
| CeO₂ | 2.000 | 0.700 | 1.545 | 0.697 | 0.200 | 1.400 | 1.597 | 0.101 | 1.303 |
| MnO₂ | 0.100 | 0.020 | 0.072 | 0.020 | — | 0.025 | 0.158 | — | 0.099 |
| V₂O₅ | 0.150 | 0.100 | 0.133 | 0.100 | 0.010 | 0.250 | 0.399 | 0.010 | 0.401 |
| Tb₄O₇ | 0.500 | 0.200 | 0.395 | 0.199 | 0.150 | 0.500 | 0.838 | — | 0.197 |
| Er₂O₃ | 0.150 | — | 0.098 | — | 0.010 | 0.300 | 0.349 | 0.010 | 0.100 |
| Co₃O₄ | — | — | — | — | — | — | — | — | 0.001 |
| Additions to | | | | | | | | | |
| the glass | | | | | | | | | |
| Fluorescent | — | — | — | — | 1.00 | 2.00 | 5.00 | 1.00 | — |
| pigment | | | | | | | | | |
| Additives | 1.50 | 1.50 | 1.50 | 1.50 | — | — | — | — | — |

Amounts of the additions are relative to the glass;
Additives were 1.0 wt.-% binder and 0.5 wt.-% pressing agent

TABLE II

| Example | 1 | 2 | 3 | 4 | 5 | 6 | 7 | 8 | 9 |
|---|---|---|---|---|---|---|---|---|---|
| Cold pressing pressure | 120 | 120 | 120 | 120 | 50 | 50 | 50 | 50 | 50 |
| (MPa) | | | | | | | | | |
| Debinding (min/° C.) | 30/440 | 30/440 | 30/440 | 30/440 | — | — | — | — | — |
| Heat treatment for | 10/670 | 10/670 | 10/670 | 10/670 | 10/670 | 10/670 | 10/670 | 10/670 | 10/670 |
| crystallization LS | | | | | | | | | |
| (min/° C.) | | | | | | | | | |
| Hot pressing | 10/750; | 10/750; | 10/750; | 10/750; | 5/750; | 3/750; | 5/750; | 3/750; | 5/750; |
| (min/° C.; MPa) | 30 | 30 | 30 | 30 | 30 | 30 | 30 | 30 | 30 |
| Density LS blank | 2.511 | 2.506 | — | 2.528 | — | — | — | — | — |
| LS content LS blank (%) | 34.7 | 33.2 | — | — | — | — | — | — | — |
| LS2 content LS blank (%) | * | 1.6 | | | | | | | |
| Heat treatment for | 10/850 | 10/850 | 10/850 | 10/850 | 7/840 | 7/840 | 7/840 | 7/840 | 7/840 |
| crystallization LS2 | | | | | | | | | |
| (min/° C.) | | | | | | | | | |
| Density LS2 blank | 2.533 | 2.524 | — | — | — | — | — | — | — |
| LS content LS2 blank (%) | 8.0 | 3.7 | — | — | — | 6.3 | 6.2 | 3.2 | — |
| LS2 content LS2 blank (%) | 35.2 | 41.4 | — | — | — | 39.4 | 38.0 | 44.5 | — |
| L | 77.23 | 86.3 | — | — | 93.05 | 79.14 | 69.59 | 90.24 | 70.54 |
| a | 6.24 | −0.52 | — | — | 0.02 | 5.57 | 9.36 | 0.36 | 6.11 |
| b | 18.64 | 15.08 | — | — | 3.42 | 25.68 | 27.3 | 5.52 | 24.85 |
| CR | 94.11 | 65.77 | — | — | 70.02 | 78.25 | 83.7 | 61.97 | 81.75 |
| σ_{B} (MPa) | 447 | 403 | — | 471 | 392 | — | — | — | — |
| K_{1c} (MPa m^{1/2}) | 2.19 | 2.45 | — | — | — | — | — | — | — |

LS—lithium metasilicate;
LS2—lithium disilicate;
* not detectable

1. Process for the preparation of a multi-coloured glass ceramic blank for dental purposes with lithium silicate as main crystal phase, in which
(a) (i) differently coloured powders of lithium silicate glasses or (ii) suspensions of differently coloured powders of lithium silicate glasses in liquid media are introduced into a mould to form a glass blank,
(b) optionally the glass blank from step (a) is compacted by pressing,
(c) the glass blank from step (a) or (b) is heat treated to obtain a glass ceramic blank with lithium silicate as main crystal phase, and
(d) the glass ceramic blank from step (c) is compacted by hot pressing.

2. Process according to claim 1, in which in step (a) powders are introduced into the mould and step (b) is carried out.

3. Process according to claim 1, in which in step (a) suspensions are introduced into the mould and the liquid media are removed.

4. Process according to claim 1, in which in step (a) the powders or suspensions are introduced into the mould in such a way that the multi-coloured glass ceramic blank prepared has a continuously changing colour.

5. Process according to claim 1, in which in step (a) the powders (i) have a particle size of from 0.5 to 150 μm, and the powders of the suspensions (ii) have a particle size of from 0.5 to 80 μm, and/or the powders (i) and the powders (ii) have an average particle size as d₅₀ value of from 5 to 30 μm.

6. Process according to claim 1, in which in step (b) the pressing is effected at a temperature of less than 60° C., and at a pressure of from 20 to 120 MPa.

7. Process according to claim 1, in which in step (c) the heat treatment is effected at a temperature of less than 700° C. and for a period of from 2 to 60 min.

8. Process according to claim 1, in which in step (d) the hot pressing is effected at a temperature of from 650 to 780° C. and at a pressure of from 5 to 50 MPa.

9. Process according to claim 1, in which in step (d) the hot pressing is effected for a period of from 0.1 to 10 min.

10. Process according to claim 1, in which in step (d) the hot pressing is effected at an atmospheric pressure of less than 0.1 bar.

11. Process according to claim 1, in which the multi-coloured glass ceramic blank has lithium metasilicate as main crystal phase.

12. Process according to claim 1, in which the multi-coloured glass ceramic blank comprises more than 10 wt.-% lithium metasilicate crystals.

13. Process according to claim 1, in which the multi-coloured glass ceramic blank has a density of from 2.4 to 2.6 g/cm³.

14. Process according to claim 1, in which the multi-coloured glass ceramic blank comprises at least one of the following components in the amounts indicated:

| Component | wt.-% |
|---|---|
| SiO₂ | 64.0 to 75.0 |
| Li₂O | 13.0 to 17.0 |
| K₂O | 0 to 5.0 |
| Al₂O₃ | 0.5 to 5.0 |
| P₂O₅ | 2.0 to 5.0 |

15. Process according to claim 1, in which the multi-coloured glass ceramic blank comprises at least one of the following components in the amounts indicated:

| Component | wt.-% |
|---|---|
| SiO₂ | 64.0 to 75.0 |
| Li₂O | 13.0 to 17.0 |
| K₂O | 0 to 5.0 |
| Al₂O₃ | 0.5 to 5.0 |
| P₂O₅ | 2.0 to 5.0 |
| ZrO₂ | 0 to 5.0 |
| MgO | 0 to 5.0 |
| SrO | 0 to 5.0 |
| ZnO | 0 to 5.0 |
| F | 0 to 1.0 |
| colouring and/or | 0 to 10.0, |
| fluorescent components | |

wherein the colouring and/or fluorescent components are selected from the group of oxides of Sn, Ce, V, Mn, Co, Ni, Cu, Fe, Cr, Tb, Eu, Er and Pr.

16. Process according to claim 1, in which in step (a) the powders (i) and the powders (ii) have an average particle size as d₅₀ value of from 10 to 20 μm.

17. Process for the preparation of a dental restoration, in which
(i) the process according to claim 1 is carried out to produce a multi-coloured glass ceramic blank,
(ii) the multi-coloured glass ceramic blank is given the shape of the dental restoration by machining, and
(iii) at least one heat treatment at a temperature of more than 750° C. is carried out.

18. Process according to claim 17, in which in step (iii) the heat treatment effects the formation of lithium disilicate as main crystal phase.

19. Process according to claim 17, in which in step (ii) the machining is effected with computer-controlled milling and/or grinding devices.

20. Process according to claim 17, in which the dental restoration is selected from the group of crowns, abutments, inlays, onlays, veneers, facets, bridges and caps.
